(19) United States
(12) Patent Application Publication
(10) Pub. No.: US 2002/0169122 A1
(43) Pub. Date: Nov. 14, 2002

(54) Chondrogenic potential of human bone marrow-derived CD105+ cells by BMP

(75) Inventors: Manas Kumar Majumdar, Burlington, MA (US); Elisabeth Ann Morris, Sherborn, MA (US)

Correspondence Address: American Home Products Corporation, 5 Giralda Farms, Madison, NJ, 07940-0874, US

(73) Assignee: Wyeth, Madison, NJ (US)

(21) Appl. No.: 10/078,808

(22) Filed: Feb. 19, 2002

Related U.S. Application Data
(60) Provisional application No. 60/271,186, filed on Feb. 23, 2001.
(60) Provisional application No. 60/333,975, filed on Nov. 29, 2001.

(51) Int. Cl. A61K 38/18

(52) U.S. Cl. 514/12

(57) ABSTRACT

Compositions of BMPs useful for cartilage repair and methods employing these compositions are disclosed. Compositions comprising non-tissue culture expanded cells isolated from bone marrow and treated with BMPs useful for cartilage repair and methods employing these compositions are also disclosed. The compositions are useful in the treatment of osteoarthritis, cartilage defects and in related tissue repair.

### CROSS REFERENCE TO RELATED APPLICATIONS

This application claims the benefit of U.S. Provisional Application No. 60/271,186, filed Feb. 23, 2001, the contents of which are incorporated by reference herein in their entirety.

### BACKGROUND OF THE INVENTION

The present invention relates to the field of tissue repair including connective tissue and cartilage repair. More specifically, the present invention relates to bone morphogenetic proteins (BMPs), and compositions which play an important role in chondrogenesis. In particular, the present invention also relates to the use of BMPs for the induction of cartilaginous tissue, such as articular cartilage, as well as the use of BMPs as therapeutics to partially block the inhibitory effect of IL-1.

The present invention further relates to the use of non-tissue culture expanded cells isolated from bone marrow for use in tissue repair. Further the present invention relates to compositions comprising non-tissue culture expanded cells isolated from bone marrow and bone morphogenetic proteins (BMPs) for the induction of cartilaginous tissue, such as articular cartilage.

Articular cartilage is avascular and aneural and consists of sparsely embedded chondrocytes in a specialized microenvironment made up of dense extracellular matrix components. The chondrocytes maintain the architecture of the cartilage through balanced anabolic and catabolic functions [Curr Opin Cell Biol 1(5), 989-94(1989)]. Cartilage injury results in the imbalance of these functions and is associated with the presence of inflammatory cytokines including interleukin-1 (IL-1) and tumor necrosis factor (TNF) [Rheumatol Int 2(2), 49-53(1982); Arthritis Rheum 29(4), 461-70(1986); Arthritis Rheum 29(2), 262-73(1986)]. Articular cartilage also has a limited spontaneous repair response when the cartilage is damaged by trauma or disease processes.

Bone marrow-derived cell components play an important role in the repair of damaged articular cartilage by being the source of progenitor cells and related growth factors that are required for their differentiation. Surgical procedures aim to supply bone marrow-derived mesenchymal precursor cells to the damaged site by penetrating the underlying subchondral bone with the hope that the surrounding environment will provide the proper stimulus for differentiation of these cells. These procedures usually result in fibrocartilage and not articular cartilage [Arthritis Rheum 42, 1331-1342(1998);in Articular Cartilage and Knee Joint Function: Basic Science and Arthroscopy (Ewing, J. W., ed), Raven Press, New York (1990)]. Repair of damaged articular cartilage requires the mobilization and differentiation of these precursor cells by cytokines and factors at the site of damage. The complex in vivo environment makes it difficult for the identification of the differentiating factors that are important in the transformation of progenitor cells into chondrocytes. [Suh et al., Operative Techniques in Orthopaedics. 7:270-278 (1997) O'Driscoll, The Journal of Bone and Joint Surgery. 80:1795-1812 (1998)]. Bone marrow consists of two cellular components: hematopoietic cells that reside in close juxtaposition with the nonhematopoietic cells. Within the nonhematopoietic compartment is a population of cells which shows multipotential mesenchymal properties and are termed multipotential mesenchymal cells (MMCs) [Majumdar et al Journal of Cellular Physiology. 185:98-106(2000);Majumdar et al Journal of Cellular Physiology. (2001) 189:275-284], mesenchymal stem cells MSCs [Pittenger et al Science. 284:143-147(1999)] 1999) or mesenchymal progenitor cells MPCs [Johnstone, et al Experimental Cell Research. 238:265-272 (1998)]. Mesenchymal precursor cells present in the bone marrow have the potential to differentiate into multiple connective tissue lineages including osteoblasts, chondrocytes, tenocytes, adipocytes and myocytes when placed in appropriate in vivo and or in vitro environments [Science 279, 1528-1530(1998); Bone 19, 421-428(1996); Bone 13, 81-95(1992); Tissue Engineering 4,415-428 (1998); Journal of Orthpedic Research 16,406-413(1998)]. These marrow-derived mesenchymal cells acquire multipotential mesenchymal characteristics only after tissue culture expansion. MMCs have been isolated from the human marrow using an immunoselection procedure that recognizes a cell surface marker, endoglin (CD 105) expressed by these cells [Majumdar et al Journal of Cellular Physiology 185:98-106(2000)].

The search for the molecule or molecules responsible for formation of bone, cartilage, tendon and other tissues present in bone and other tissue extracts has led to the discovery of a novel set of molecules called the Bone Morphogenetic Proteins (BMPs). The structures of several proteins, designated BMP-1 through BMP-16, among others have previously been elucidated. Bone morphogenetic proteins (BMPs), TGF-β and insulin-like growth factors have been shown to promote chondrogenesis or demonstrate chondrogenic effect both in vivo and in vitro [J Cell Physiol 185(1), 98-106(2000); Bone 19(1 Suppl), 1S-12S(1996); Clin Orthop (367 Suppl), S186-203 (1999)]. BMPs are secreted molecules of the TGF-β superfamily of growth and differentiation factors that were originally detected in and purified from demineralized bone [Proc Natl Acad Sci U S A 85(24), 9484-8(1998)]. Twenty mammalian BMPs have been identified, and three type II receptors have been shown to bind BMPs [Trends Genet 10(1), 16-21(1994)]. BMP binding leads to dimerization of type I and II receptors prior to phosphorylation and signaling through the Smad pathway [Bone 19(6), 569-74(1996)]. BMPs have been shown to function as key regulators in cartilage and bone development [Annu Rev Biochem 67, 753-91(1998)], and also function in repair and remodeling of the adult skeletal system [Genes Dev 3(11), 1657-68(1989); J Bone Miner Res 14(10), 1734-41 (1999); The Journal of Bone and Joint Surgery 82-A(2), 151-160(2000)].

Sox-9, a transcription factor, has been shown to be an important downstream mediator of the BMP-2 signaling pathway [The Journal of Bone and Joint Surgery 82-A(2), 151-160(2000)]. Sox-9 is characterized by the presence of a 79 amino acid high mobility group-type DNA-binding domain with high homology to that of sex-determining region Y (Sry) [Curr Opin Genet Dev 7(3), 338-44(1997)]. Sox-9 is expressed during embryonic development in a pattern that closely parallels that of the gene for Col2A1 [Dev Dyn 209(4), 377-86(1997); Dev Biol 183(1), 108-21(1997)] and cartilage matrix synthesis [Genes Dev 3(11), 1657-68(1989); J Bone Miner Res 14(10), 1734-41(1999); The Journal of Bone and Joint Surgery 82-A(2), 151-160(2000); Science 289(5477), 313-6(2000); J Biol Chem 275(24), 17937-45(2000); Curr Opin Genet Dev 7(3), 338-44(1997); Dev Dyn 209(4), 377-86(1997); Dev Biol 183(1), 108-21(1997); Nat Genet 16(2), 174-8(1997)], suggesting a role for Sox-9 in chondrogenesis and skeletogenesis. It has been shown that upregulation of Sox-9 enhances the expression of both Col2A1 and aggrecan in immortalized cell lines The Journal of Bone and Joint Surgery 82-A(2), 151-160(2000). Proinflammatory molecules including IL-1 and TNF inhibit the expression of Col2A1 and aggrecan The Journal of Bone and Joint Surgery 82-A(2), 151-160(2000); J Clin Invest 82(6), 2026-37(1998); Biochim Biophys Acta 1052(3), 366-78(1990); J Cell Physiol 166(2), 351-9(1996)]. It has been reported that the inhibitory effects of these inflammatory cytokines are mediated by the down regulation of Sox-9 [J Biol Chem 275(5), 3687-92(2000)]. The inhibitory effects IL-1 and TNF, present at elevated levels in osteoarthritis and rheumatoid arthritis have been implicated in the breakdown of cartilage in these disease states [Rheumatol Int 2(2), 49-53 (2000); Arthritis Rheum 29(4), 461-70(1986); Arthritis Rheum 29(2), 262-7391986); J Biol Chem 275(5), 3687-92(2000)].

### SUMMARY OF THE INVENTION

By the present invention, Applicants have demonstrated that BMP-2 and BMP-9 promote chondrogenic differentiation of human mesenchymal precursor cells. Applicants have further demonstrated that the chondrogenic potential of these BMPs were able to overcome the inflammatory effect of IL-1. The ability of the BMPs to stimulate matrix synthesis by articular chondrocytes and maintain chondrocyte phenotype suggest important applications including cartilage defect repair and prevention/reversal of osteoarthritis, chondrocyte phenotype. These BMPs may be particularly useful for cartilage differentiation, growth, maintenance and repair. The present invention is therefore directed to composition and methods comprising BMPs in chondrogenesis. The present invention is further directed to the use of BMPs to block or partially block the inflammatory effect of IL-1. The BMPs and other proteins useful in the invention are further described below.

In the present invention, compositions containing a BMP- are administered to a patient in need of cartilage repair, or having a disease or defect involving cartilaginous tissue, such as osteoarthritis. In a preferred embodiment, the present invention comprises compositions comprising an effective amount of BMP-2 or BMP-9.

In the compositions, the protein may be admixed with a pharmaceutically acceptable vehicle. In a particular embodiment, the composition may additionally include one or more additional transforming growth factor-β proteins or bone morphogenetic proteins. The composition comprising both a BMP related protein and another TGF-β or BMP may be useful for especially useful for the treatment of articular cartilage, in which the articular surface, cartilage, subchondral bone and/or tidemark interface between cartilage and bone may need to be repaired.

The present invention also includes methods for cartilaginous tissue healing and tissue repair, for treating osteoarthritis, or other cartilage defects, and for inducing cartilaginous tissue formation in a patient in need of same, comprising administering to said patient an effective amount of a BMP composition. In preferred embodiments the composition utilized in the methods comprises BMP-2 and/or BMP-9. The invention also includes heterodimeric protein molecules comprising one monomer having the amino acid sequence of a protein which is useful for the induction of chondrocytes or cartilaginous tissue, and one monomer having the amino acid sequence of another protein of the TGF-β subfamily.

The present invention is further directed to compositions comprising non-tissue culture expanded cells isolated from bone marrow which have chondrogenic potential. In a preferred embodiment the non-tissue culture expanded cells are CD105+ cells. In a further embodiment the composition of the invention comprises non-tissue culture expanded cells isolated from human bone marrow and a protein which induces the formation of cartilage and/or bone. These cells isolated from bone marrow and non-tissue culture expanded demonstrate chondrogenic potential when treated with BMP.

In preferred embodiments, the active agent for treatment of non-tissue culture expanded cells and for use in other embodiments of the invention include one or more proteins selected from the group of proteins known as the Transforming Growth Factors-Beta (TGF-β) superfamily of proteins, preferably selected from the Bone Morphogenetic Proteins (BMPs), the Growth and Differentiation Factors (GDFs), as well as other proteins, as described more fully herein. Osteogenic proteins, DNA sequences, compositions and methods for producing them, useful in the present invention, are those comprising the BMP proteins BMP-2, BMP-3, BMP-4, BMP-5, BMP-6 and BMP-7, disclosed for instance in U.S. Pat. Nos. 5,108,922; 5,013,649; 5,116,738; 5,106,748; 5,187,076, 5,459,047, 5,849,880; and 5,141,905; BMP-8, disclosed in PCT publication WO91/18098; and BMP-9, disclosed in PCT publication WO93/00432, BMP-10, disclosed in PCT application WO94/26893; BMP-11, disclosed in PCT application WO94/26892, or BMP-12 or BMP-13, disclosed in PCT application WO95/16035, or BMP-15, disclosed in PCT application WO96/36710 or BMP-16, disclosed in co-pending patent application Ser. No. 08/715/202, filed Sep. 18, 1996. In a preferred embodiment the BMP is selected from the group consisting of BMP-2 and BMP-9.

Other DNA molecules and the proteins which they encode which may also be useful include those encoding Vgr-2, and any of the growth and differentiation factors [GDFs], including those described in PCT applications WO94/15965; WO94/15949; WO95/01801; WO95/01802; WO94/21681; WO94/15966; and others. Also useful in the present invention may be BIP, disclosed in WO94/01557; and MP52, disclosed in PCT application WO93/16099. The disclosures of all of the above applications are hereby incorporated by reference for the disclosure contained therein.

Other DNA molecules and the proteins which they encode which may be useful including growth factors such as epidermal growth factor (EGF), fibroblast growth factor (FGF), transforming growth factor (TGF-α and TGF-β), hedgehog proteins such as sonic, indian and desert hedgehog, parathyroid hormone and parathyroid hormone related peptide, cadherins, activins, inhibins, and IGF, FSH, frizzled, frzb or frazzled proteins, PDGF and other endothelial growth factors, BMP binding proteins such as chordin and fetuin, estrogen and other steroids as well as truncated versions thereof, and transcription factors such as wnt proteins, mad genes and cbfa.

The disclosures of the above identified applications are hereby incorporated herein by reference. The unique inductive activities of these proteins, along with their presence in bone, suggests that they are important regulators of bone and cartilage repair processes, and may be involved in the normal maintenance of bone tissue.

The isolated cells of the invention may be treated with the BMP or other cartilage inducing protein. In further embodiments the DNA sequences encoding the BMP proteins may be incorporated into the cells using methods known to those skilled in the art.

Cells directly isolated from the marrow without expansion are preferable for therapeutic purposes for several reasons. First, selection based on adherence preferentially chooses a subpopulation of cells demonstrating a characteristic which has never been shown to necessarily correlate with chondrogenic potential. The liklihood of discarding a potential important subpopulation of cells with chondrogenic capabilities based on their inability to adhere is diminished. It is possible that in vitro responses to differentiation factors during culture expansion may alter cell surface characteristics rendering the cells immunogenic to the host, and resulting in a graft versus host response after transplantation. Finally, by the present invention it has been demonstrated that the chondrogenic differentiation of CD105⁺ cells is not dependent on culture and/or expansion of the cells. Based on chondrogenic differentiation of human bone marrow-derived CD 105⁺ cells in a 3-dimensional matrix in the presence of BMPs in serum-free conditions the invention therefore features a clinical transplant protocol employing bone marrow-derived autologous cells transplanted for the repair of articular cartilage. This protocol eliminates the extended, expensive and laborious culture expansion of the cells.

The present invention therefore further features CD105⁺ cells isolated from human marrow- and directly encapsulated in a 3-dimensional matrix of alginate and cultured in a serum-free medium. The compositions of the invention may therefore further comprise a pharmaceutically acceptable vehicle or suitable matrix.

The present invention also includes methods for cartilaginous tissue healing and tissue repair, for treating osteoarthritis, or other cartilage defects, and for inducing cartilaginous tissue formation in a patient in need of same, comprising administering to said patient an effective amount of a composition of the invention comprising non-tissue culture expanded cells isolated from bone marrow and a bone and/or cartilage inducing protein. In preferred embodiments the composition comprises CD 105+ cells and BMP.

In a preferred embodiment, the method of the present invention comprises administering compositions comprising these CD 105+ cells and an effective amount of BMP-2 or BMP-9. In another embodiment, this method comprises administering to said patient simultaneously with the cells or subsequently an effective amount of a composition comprising BMP-2 or BMP-9.

Various clinical applications have been proposed using primary stem and progenitor cells [Fuchs et al Cell. 100:143-155 (2000)]. Mesenchymal cell therapies have been proposed for various tissue repair with culture-expanded cells [Caplan Journal of Orthopaedic Research. 9:641-650(1991). The present invention widens the clinical applications of cell-based tissue repair, procedures which minimize the in vitro manipulation of these cells would be advantageous. The differentiation potential of mesenchymal cells without culture expansion as shown by the present invention provide for clinical treatments of connective tissue diseases.

### DESCRIPTION OF THE DRAWINNG

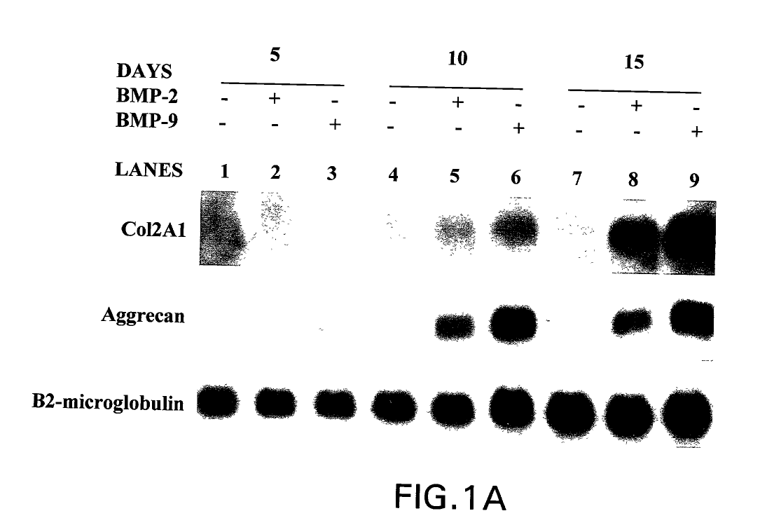

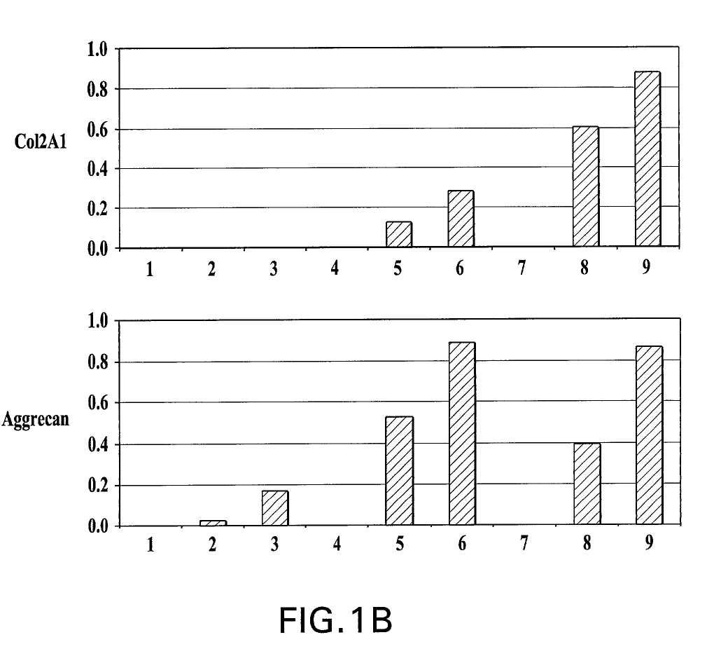

FIG. 1 is directed to the induction of the expression of chondrogenic markers in a time-dependant manner by BMP-2 and BMP-9. FIG. 1A, total RNA was isolated and subjected to Northern analysis with Col2A1, aggrecan and Sox-9 probes as well as a β2-microglobulin probe as a loading control. FIG. 1B, quantitation of Col2A1, aggrecan and Sox-9 signals by scanning densitometry is shown. Lanes 1, 4 and 7-untreated cells; lanes 2, 5 and 8-rhBMP-2 treated cells; lanes 3, 6 and 9-rhBMP-9 treated cells.

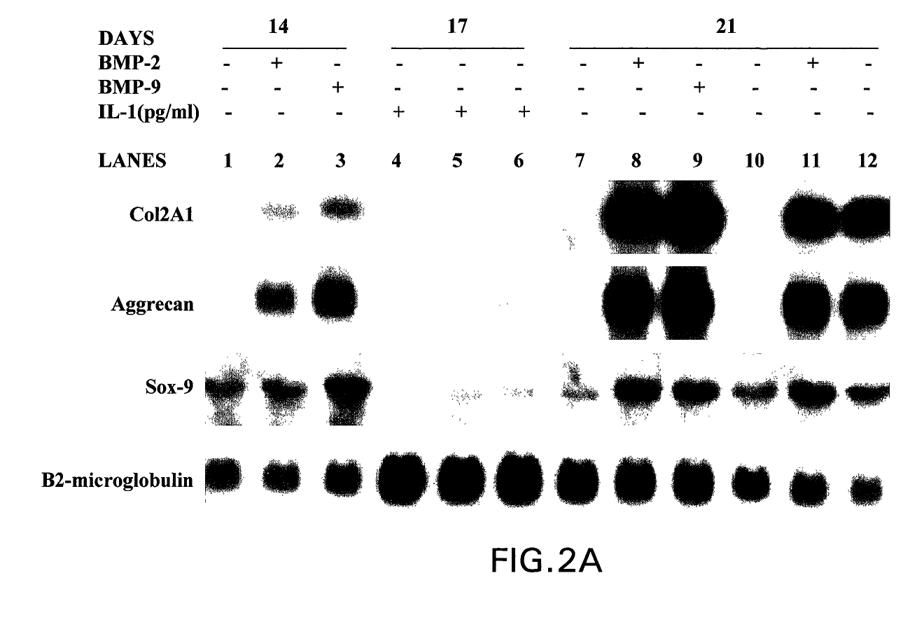

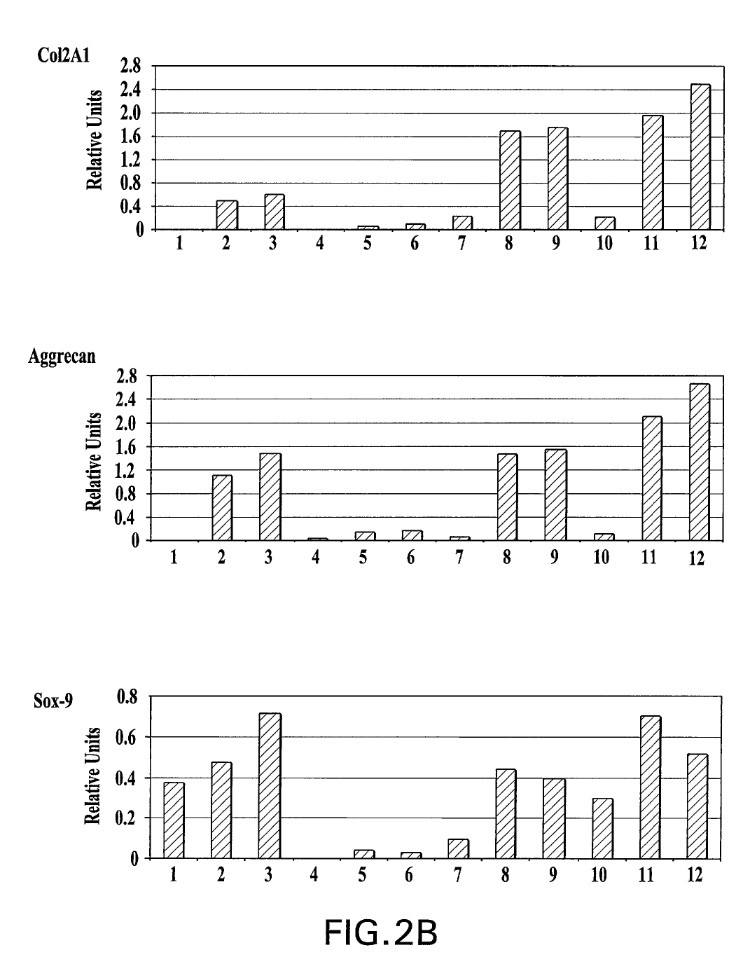

FIG. 2 indicates that BMP-2 and BMP-9 are able to reverse the expression of chondrogenic markers after IL-1 withdrawal. FIG. 2A, total RNA was isolated and subjected to Northern analysis with Col2A1, aggrecan and Sox-9 probes as well as a β2-microglobulin probe as a loading control. Expression of Col2A1, aggrecan and Sox-9 after 14 days in culture are demonstrated (lanes 1-3). Cell aliquots from the cultures were removed, washed and cultured for 72 h in media with IL-1 at 200 pg/ml (lanes 4-6). Cell aliquots of IL-1 treated cells were removed, washed and cultured with or without BMPs for an additional 96 h (lanes 7-9). Parallel cultures with or without BMPs were also maintained for the total culture period of 21 days (lanes 10-12). FIG. 2B, quantitation of Col2A1, aggrecan and Sox-9 signals by scanning densitometry is shown.

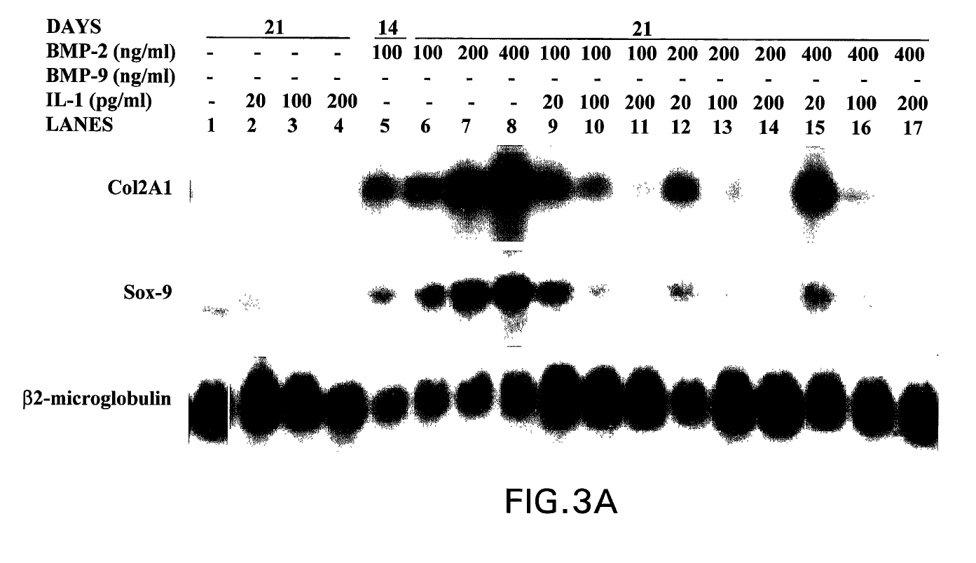

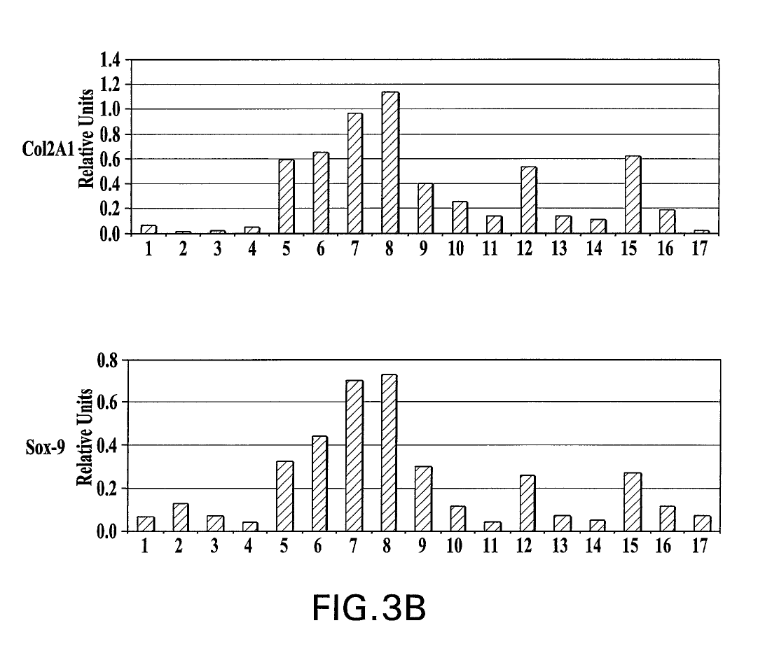

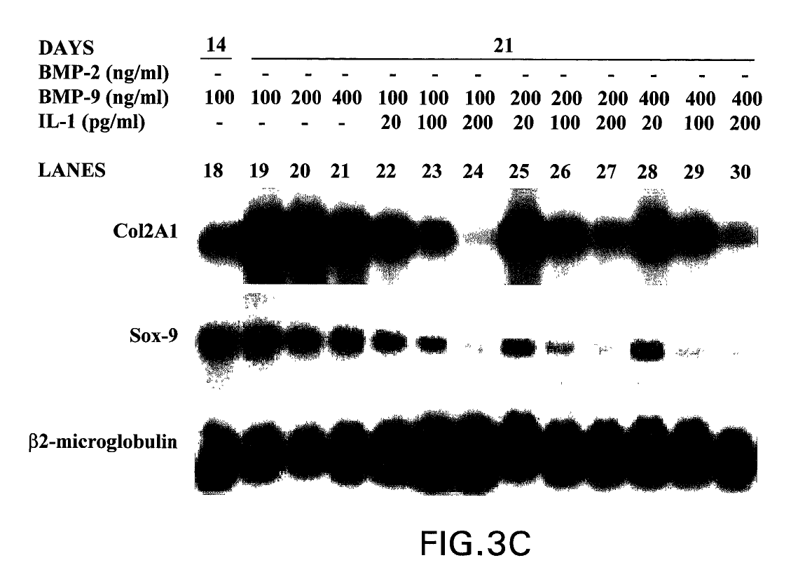

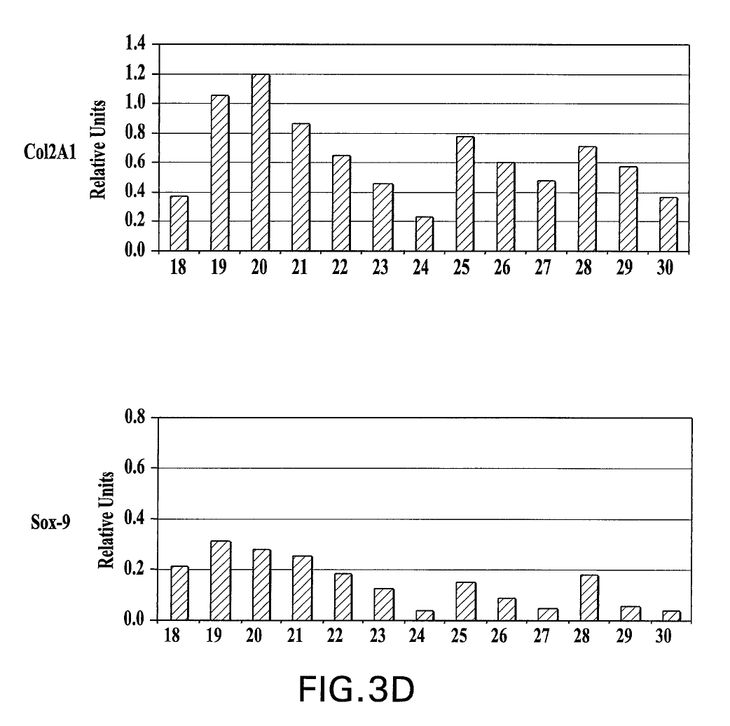

FIG. 3 indicates the ability of BMP-2 and BMP-9 to overcome the inhibitory effect of IL-1. FIGS. 3A and 3C, total RNA was isolated and subjected to northern analysis with Col2A1 and Sox-9 probes as well as a β2-microglobulin probe as a loading control. Cells untreated for 21 days (lane 1) and untreated cells cultured for 14 days were treated with IL-1 for the next 7 days (lanes 2-4). Cells treated with BMP-2 for 14 days (lanes 5), aliquots of the BMP-2 treated cells were either cultured for an additional 7 days in increasing concentrations of BMP-2 (lanes 6-8) or in BMP-2 and IL-1 together (lanes 9-17). Cells treated with BMP-9 for 14 days (lanes 18), aliquots of the BMP-9 treated cells were either cultured for an additional 7 days in increasing concentrations of BMP-9 (lanes 19-21), or in BMP-9 and IL-1 together (lanes 22-30). FIGS. 3B and 3D, quantitation of Col2A1 and Sox-9 signals by scanning densitometry is shown.

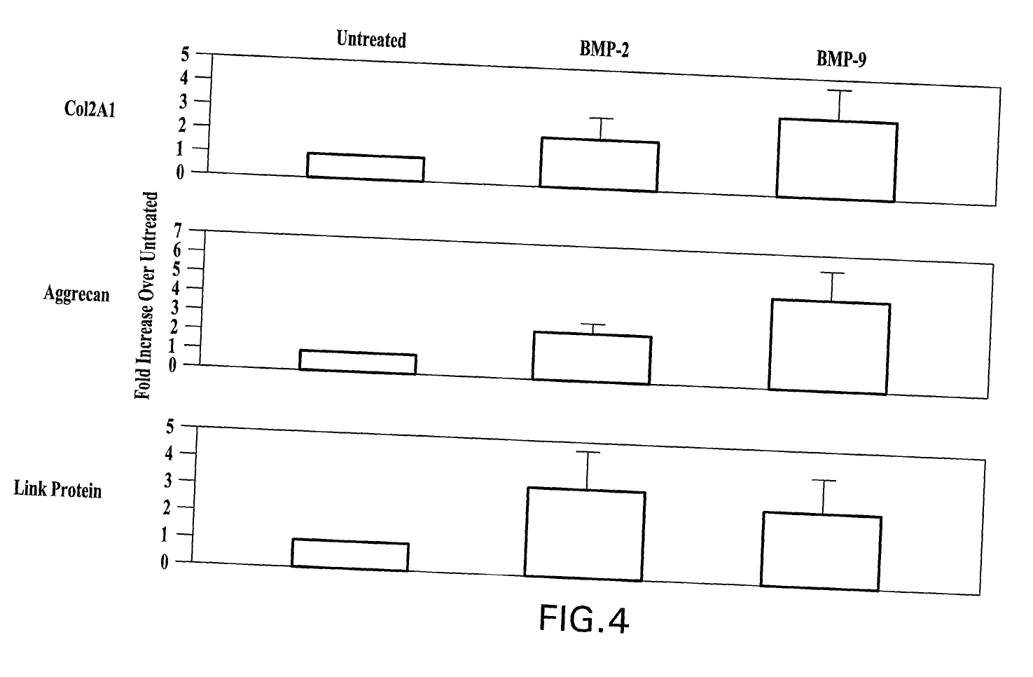

FIG. 4: Gene expression of cartilage specific markers by CD 105⁺ cells in alginate cultures. CD105⁺ cells isolated from human bone marrow were encapsulated in alginate and cultured in a serum-free media (untreated) supplemented with BMP-2 or BMP-9 for 3 weeks. RT-PCR elisa for type II collagen, aggrecan and link protein was performed on RNA extracted from the cells. The bars represent the mean (+SEM) from 3 donors.

### DETAILED DESCRIPTION OF THE INVENTION

The invention is directed to compositions comprising BMPs which promote chondrogenic differentiation. These compositons are able to maintain the expression of chondrocyte specific extracellular matrix molecules in the presence of osteoarthritis-related physiological levels of IL-1. The invention is further directed to methods utilzing these compositions. Preferred BMPs for the compositions and methods are BMP-2 and BMP-9. The DNA encoding and amino acid sequences of BMP-2 and methods for preparing the same are described for example in U.S. Pat. No. 5,013,649, the disclosure of which is incorporated herein by reference. The DNA encoding and amino acid sequences of BMP-9 are disclosed in WO93/00432, the disclosure of which is incorporated herein by reference.

The present invention is also directed to compositions comprising non-tissue culture expanded cells isolated from bone marrow which have chondrogenic potential. In a preferred embodiment the non-tissue culture expanded cells are CD105+ cells. In a further embodiment the composition of the invention comprises non-tissue culture expanded cells isolated from human bone marrow and a protein which induces the formation of cartilage and/or bone. These cells isolated from bone marrow and non-tissue culture expanded demonstrate chondrogenic potential when treated with BMP. By the present invention, Applicant have shown these cells have the potential to be the source of precursor cells important for clinical treatments of connective tissue diseases including cartilage repair. The isolated cells may therefore be treated with the BMP proteins. In further embodiments the sequences encoding the BMPs may be incorporated into the cells.

The compositions and methods of the present invention find application in the induction of cartilaginous tissue or other tissue formation in circumstances where such tissue is not normally formed, and has application in the healing of cartilage, for example articular cartilage tears, deformities and other cartilage defects in humans and other animals. Such a preparation employing a cartilaginous tissue inducing protein may have prophylactic use in preventing damage to cartilaginous tissue, as well as use in the improved fixation of cartilage to bone or other tissues, and in repairing defects to cartilage tissue. De novo cartilaginous tissue formation induced by a composition of the present invention contributes to the repair of congenital, trauma induced, or other cartilage defects of other origin, and is also useful in surgery for attachment or repair of cartilage. The compositions of the invention may also be useful in the treatment of arthritis and other cartilage defects. The compositions of the present invention can also be used in other indications wherein it is desirable to heal or regenerate cartilage tissue. Such indications include, without limitation, regeneration or repair of injuries to the articular cartilage. The compositions of the present invention may provide an environment to attract cartilage-forming cells, stimulate growth of cartilage-forming cells or induce differentiation of progenitors of cartilage-forming cells.

The proteins useful in the methods of the present invention are capable of inducing the formation of cartilaginous tissue. By cartilaginous tissue, it is meant chondrocytes, and tissue which is formed by chondrocytes, which demonstrate the histological and compositional characteristics of cartilage. These proteins may be further characterized by the ability to demonstrate cartilaginous tissue formation activity in the assays described below. It is contemplated that these proteins may have ability to induce the formation of other types of tissue, such as tendon and ligament.

The compositions for inducing cartilaginous tissue formation of the present invention may comprise an effective amount of a cartilaginous tissue inducing protein. In preferred embodiments, the active agent is one or more proteins selected from the group of proteins known as the Transforming Growth Factors-Beta (TGF-β) superfamily of proteins, preferably selected from the Bone Morphogenetic Proteins (BMPs), the Growth and Differentiation Factors (GDFs), as well as other proteins, as described more fully herein. Osteogenic proteins, DNA sequences, compositions and methods for producing them, useful in the present invention, are those comprising the BMP proteins BMP-2, BMP-3, BMP-4, BMP-5, BMP-6 and BMP-7, disclosed for instance in U.S. Pat. Nos. 5,108,922; 5,013,649; 5,116,738; 5,106,748; 5,187,076, 5,459,047, 5,849,880; and 5,141,905; BMP-8, disclosed in PCT publication WO91/18098; and BMP-9, disclosed in PCT publication WO93/00432, BMP-10, disclosed in PCT application WO94/26893; BMP-11, disclosed in PCT application WO94/26892, or BMP-12 or BMP-13, disclosed in PCT application WO95/16035, or BMP-15, disclosed in PCT application WO96/36710 or BMP-16, disclosed in co-pending patent application Ser. No. 08/715/202, filed Sep. 18, 1996. Preferred BMPs for the compositions and methods are BMP-2 and BMP-9. The DNA encoding and amino acid sequences of BMP-2 and methods for preparing the same are described for example in U.S. Pat. No. 5,013,649, the disclosure of which is incorporated herein by reference. The DNA encoding and amino acid sequences of BMP-9 are disclosed in WO93/00432, the disclosure of which is incorporated herein by reference.

Other DNA molecules and the proteins which they encode which may also be useful include those encoding Vgr-2, and any of the growth and differentiation factors [GDFs], including those described in PCT applications WO94/15965; WO94/15949; WO95/01801; WO95/01802; WO94/21681; WO94/15966; and others. Also useful in the present invention may be BIP, disclosed in WO94/01557; and MP52, disclosed in PCT application WO93/16099. The disclosures of all of the above applications are hereby incorporated by reference for the disclosure contained therein.

It is expected that the proteins act in concert with or perhaps synergistically with other related proteins and growth factors. Other DNA molecules and the proteins which they encode which may be useful, in addition to DNA encoding a BMP protein, include DNA molecules encoding other therapeutically useful agents including growth factors such as epidermal growth factor (EGF), fibroblast growth factor (FGF), FGF-4, transforming growth factor (TGF-α and TGF-β), leukemia inhibitory factor (LIF/HILDA/DIA), insulin-like growth factors (IGF-I and IGF-II), interleukins such as IL-11, hedgehog proteins such as sonic, indian and desert hedgehog, parathyroid hormone and parathyroid hormone related peptide, cadherins, activins, inhibins, and IGF, FSH, frizzled,frzb orfrazzled proteins, PDGF and other endothelial growth factors, BMP binding proteins such as chordin and fetuin, estrogen and other steroids as well as truncated versions thereof, and transcription factors such as wnt proteins, mad genes and cbfa. Portions of these agents may also be used in compositions of the present invention. Such a composition may be useful for treating defects of the junction between cartilage, and bone form simultaneously at contiguous anatomical locations, and may be useful for regenerating tissue at the site of cartilage attachment to bone.

The disclosures of the above identified applications are hereby incorporated herein by reference. The unique inductive activities of these proteins, along with their presence in bone, suggests that they are important regulators of bone and cartilage repair processes, and may be involved in the normal maintenance of bone tissue.

The cartilaginous tissue-inducing proteins provided herein also include factors encoded by the sequences similar to those of the naturally-occurring protein, but into which modifications are naturally provided (e.g. allelic variations in the nucleotide sequence which may result in amino acid changes in the polypeptide) or deliberately engineered. For example, synthetic polypeptides may wholly or partially duplicate continuous sequences of the amino acid residues of the proteins. These sequences, by virtue of sharing primary, secondary, or tertiary structural and conformational characteristics with cartilaginous tissue growth or maintenance factor polypeptides of naturally-occurring proteins may possess cartilaginous or other tissue growth or maintenance factor biological properties in common therewith. Thus, they may be employed as biologically active substitutes for naturally-occurring cartilaginous tissue inducing polypeptides, and cartilaginous tissue maintenance polypeptides in therapeutic compositions and processes.

Other specific mutations of the sequences of cartilaginous tissue inducing proteins described herein involve modifications of glycosylation sites. These modifications may involve O-linked or N-linked glycosylation sites. For instance, the absence of glycosylation or only partial glycosylation results from amino acid substitution or deletion at asparagine-linked glycosylation recognition sites. The asparagine-linked glycosylation recognition sites comprise tripeptide sequences which are specifically recognized by appropriate cellular glycosylation enzymes. These tripeptide sequences may be asparagine-X-threonine, asparagine-X-serine or asparagine-X-cysteine, where X is usually any amino acid except proline. A variety of amino acid substitutions or deletions at one or both of the first or third amino acid positions of a glycosylation recognition site (and/or amino acid deletion at the second position) results in non-glycosylation at the modified tripeptide sequence. Additionally, bacterial expression of protein will also result in production of a non-glycosylated protein, even if the glycosylation sites are left unmodified.

The preparation and formulation of such physiologically acceptable protein compositions, having due regard to pH, isotonicity, stability and the like, is within the skill of the art. The therapeutic compositions are also presently valuable for veterinary applications due to the lack of species specificity in TGF-β proteins. Particularly domestic animals and thoroughbred horses in addition to humans are desired patients for such treatment with the compositions of the present invention.

The therapeutic method includes administering the composition topically, systemically, or locally as an injectable and/or implant or device. When administered, the therapeutic composition for use in this invention is, of course, in a pyrogen-free, physiologically acceptable form. Further, the composition may desirably be encapsulated or injected in a viscous form for delivery to the site of tissue damage. Topical administration may be suitable for wound healing and tissue repair. Therapeutically useful agents other than the proteins which may also optionally be included in the composition as described above, may alternatively or additionally, be administered simultaneously or sequentially with the composition in the methods of the invention. In addition, the compositions of the present invention may be used in conjunction with presently available treatments for cartilage injuries, such as suture (e.g., vicryl sutures or surgical gut sutures, Ethicon Inc., Somerville, N.J.) or cartilage allograft or autograft, in order to enhance or accelerate the healing potential of the suture or graft. For example, the suture, allograft or autograft may be soaked in the compositions of the present invention prior to implantation. It may also be possible to incorporate the protein or composition of the invention onto suture materials, for example, by freeze-drying.

As described above, the compositions of the invention may be employed in methods for treating a number of cartilage defects, such as the regeneration of cartilaginous tissue in areas of cartilage damage, to assist in repair of tears of cartilage tissue, and various other types of tissue defects or wounds. These methods, according to the invention, entail administering to a patient needing such cartilaginous tissue or other tissue repair, a composition comprising an effective amount of a cartilaginous tissue inducing protein, such as described in WO95/16035, the disclosure of which is hereby incorporated by reference. These methods may also entail the administration of a cartilaginous tissue inducing protein in conjunction with at least one of the BMP proteins described above.

In another embodiment, the methods may entail administration of a heterodimeric protein in which one of the monomers is a cartilaginous tissue inducing BMP polypeptide and the second monomer is a member of the TGF-β superfamily of growth factors. In addition, these methods may also include the administration of a cartilaginous tissue inducing protein with other growth factors including EGF, FGF, TGF-α, TGF-β, and IGF.

Thus, a further aspect of the invention is a therapeutic method and composition for repairing cartilaginous tissue, for repairing cartilage as well as treating arthritis and other conditions related to arthritis defects. Such compositions comprise a therapeutically effective amount of one or more cartilaginous tissue inducing proteins, such as BMP-2 or BMP-9, in admixture with a pharmaceutically acceptable vehicle, carrier or matrix.

Culture-expanded MMCs could be engineered to deliver chondrogenic growth factors to the site of articular cartilage repair. Therefore, the combination of MMCs and BMPs may provide and significantly improve clinical cartilage repair procedures.

The dosage regimen for embodiments of the invention will be determined by the attending physician considering various factors which modify the action of the composition, e.g., amount of cartilaginous tissue desired to be formed, the site of cartilaginous tissue damage, the condition of the damaged cartilaginous tissue, the size of a wound, type of damaged tissue, the patient's age, sex, and diet, the severity of any infection, time of administration and other clinical factors. The dosage may vary with the type of matrix used in the reconstitution and the types of additional proteins in the composition. The addition of other known growth factors, such as IGF-I (insulin like growth factor I), to the final composition, may also affect the dosage. In general, the amount of recombinant BMP protein useful for inducing formation of cartilaginous tissue will be in an amount of about 1 to about 100 ug for a defect of approximately 20 cc in volume. In general, the amount of recombinant BMP protein useful for inducing maintenance of cartilaginous tissue will be in an amount of about 1 to about 1000 ng per ml of solution.

The identification of patients needing treatment for various conditions including articular cartilage damage may be accomplished by procedures which are well known in the art. These procedures include measurement of bone mass/density using dual-energy X-ray absorptiometry (DEXA), Kilgus et al., J. Bone & Joint Surgery, 75-B:279-287 (1992); Markel et al., Acta Orthop Scand, 61:487-498 (1990); and quantitative computed tomography (QCT), Laval-Jeantet et al., J Comput Assist Tomogr, 17:915-921 (1993); Markel, Calcif Tissue Int, 49:427-432 (1991); single-photon absorptiometry, Markel et al. Calcif Tissue Int, 48:392-399 (1991); ultrasound transmission velocity (UTV); Heaney et al., JAMA, 261:2986-2990 (1989); Langton et al., Clin Phys Phvsiol Meas, 11:243-249 (1990); and radiographic assessment, Gluer et al., J Bone & Mineral Res, 9:671-677 (1994). Other methods of identification are known to those skilled in the art. The above publications are hereby incorporated by reference herein.

Progress can be monitored by periodic assessment of cartilaginous tissue formation, or cartilaginous tissue growth and/or repair. The progress can be monitored by methods known in the art, for example, X-rays, arthroscopy, histomorphometric determinations and tetracycline labeling.

Cells directly isolated from the bone marrow without expansion have several therapeutic advantages. Selection based on adherence preferentially chooses a subpopulation of cells demonstrating a characteristic which has never been shown to necessarily correlate with chondrogenic potential. The liklihood of discarding a potential important subpopulation of cells with chondrogenic capabilities based on their inability to adhere is diminished. In vitro responses to differentiation factors during culture expansion may alter cell surface characteristics rendering the cells immunogenic to the host, and resulting in a graft versus host response after transplantation. Finally, by the present invention it has been demonstrated that the chondrogenic differentiation of CD 105⁺ cells is not dependent on culture and/or expansion of the cells. Based on chondrogenic differentiation of human bone marrow-derived CD 105⁺ cells in a 3-dimensional matrix in the presence of BMPs in serum-free conditions the invention therefore features a clinical transplant protocol employing bone marrow-derived autologous cells transplanted for the repair of articular cartilage. This protocol eliminates the extended, expensive and laborious culture expansion of the cells.

The present invention further features non-tissue culture expanded CD105⁺ cells isolated from human marrow- and directly encapsulated in a 3-dimensional matrix of alginate and cultured in a serum-free medium. A further embodiment therefore includes a suitable matrix.

The present invention includes methods for cartilaginous tissue healing and tissue repair, for treating osteoarthritis, or other cartilage defects, and for inducing cartilaginous tissue formation in a patient in need of same, comprising administering to said patient an effective amount of a composition of the invention comprising non-tissue culture expanded cells isolated from bone marrow and a bone and/or cartilage inducing protein. In preferred embodiments the composition comprises non-tissue culture expanded CD105⁺ cells and BMP. In a preferred embodiment, the present invention comprises compositions comprising CD 105⁺ cells and an effective amount of BMP-2 or BMP-9. This method comprises administering to said patient simultaneously with the cells or subsequently an effective amount of a composition comprising BMP-2 or BMP-9.

Various clinical applications have been proposed using primary stem and progenitor cells [Fuchs et al Cell. 100:143-155 (2000)]. Mesenchymal cell therapies have been proposed for various tissue repair with culture-expanded cells [Caplan Journal of Orthopaedic Research. 9:641-650(1991). The present invention widens the clinical applications of cell-based tissue repair, procedures which minimize the in vitro manipulation of these cells is advantageous. The differentiation potential of mesenchymal cells without culture expansion as shown by the present invention provide for clinical treatments of connective tissue diseases.

The compositions of the invention may include an appropriate matrix and/or sequestering agent as a carrier. For instance, the matrix may support the composition or provide a surface for cartilaginous tissue formation and/or other tissue formation. The matrix may provide slow release of the protein and/or the appropriate environment for presentation thereof. The sequestering agent may be a substance which aids in ease of administration through injection or other means, or may slow the migration of protein from the site of application.

The choice of a carrier material is based on biocompatibility, biodegradability, mechanical properties, cosmetic appearance and interface properties. The particular application of the compositions will define the appropriate formulation. Potential matrices for the compositions may be biodegradable and chemically defined. Further matrices are comprised of pure proteins or extracellular matrix components. Other potential matrices are non-biodegradable and chemically defined. Preferred matrices include collagen-based materials, including sponges, such as Helistat® (Integra LifeSciences, Plainsboro, N.J.), or collagen in an injectable form, as well as sequestering agents, which may be biodegradable, for example hyaluronic acid derived. Biodegradable materials, such as cellulose films, or surgical meshes, may also serve as matrices. Such materials could be sutured into an injury site, or wrapped around the cartilage.

Another preferred class of carrier are polymeric matrices, including polymers of poly(lactic acid), poly(glycolic acid) and copolymers of lactic acid and glycolic acid. These matrices may be in the form of a sponge, or in the form of porous particles, and may also include a sequestering agent. Suitable polymer matrices are described, for example, in WO93/00050, the disclosure of which is incorporated herein by reference.

Additional optional components useful in the practice of the subject application include, e.g. cryogenic protectors such as mannitol, sucrose, lactose, glucose, or glycine (to protect the protein from degradation during lyophilization), antimicrobial preservatives such as methyl and propyl parabens and benzyl alcohol; antioxidants such as EDTA, citrate and BHT (butylated hydroxytoluene); and surfactants such as poly(sorbates) and poly(oxyethylenes).

Preferred families of sequestering agents include blood, fibrin clot and/or cellulosic materials such as alkylcelluloses (including hydroxyalkylcelluloses), including methylcellulose, ethylcellulose, hydroxyethylcellulose, hydroxypropylcellulose, hydroxypropyl-methylcellulose, and carboxymethylcellulose, the most preferred being cationic salts of carboxymethylcellulose (CMC). Other preferred sequestering agents include hyaluronic acid, sodium alginate, poly(ethylene glycol), polyoxyethylene oxide, carboxyvinyl polymer and poly(vinyl alcohol). The amount of sequestering agent useful herein is 0.5-20 wt %, preferably 1-10 wt % based on total formulation weight, which represents the amount necessary to prevent desorbtion of the protein from the polymer matrix and to provide appropriate handling of the composition, yet not so much that the progenitor cells are prevented from infiltrating the matrix, thereby providing the protein the opportunity to assist the activity of the progenitor cells.

In particular embodiments of the invention, components of the composition may be encapsulated in a resorbable polymer delivery system, such as polylactic acid, polyglycolic acid or copolymers thereof, polyorthoesters, polyorthocarbonates, and other polymers. Suitable polymers are disclosed for example in EP 0145240, the disclosure of which is hereby incorporated by reference. Alternatively, the BMP may be encapsulated in liposomes For example, liposome delivery of TGF-β protein is described in U.S. Pat. Nos. 5,206,023, 5,270,300; and 5,368,858, the disclosure of each of which are hereby incorporated by reference. Both of these delivery systems may be modified to provide for release of BMP at a later time, or over a more sustained time period, allowing for the beneficial effects of the BMP on chondrocyte and cartilage maintenance to act complementary to the beneficial effects of the BMP on induction of chondrocytes and cartilaginous tissue.

The proteins and compositions of the present invention may also be useful for treating cell populations, such as embryonic cells or stem cell populations, to enhance or enrich the growth, differentiation and/or maintenance of the cells. The treated cell populations may be useful for gene therapy applications.

The following examples illustrate practice of the present invention in demonstrating BMP promotion of chondrogenic differentiation of human mesenchymal precursor cells and the ability of these BMPs to overcome the inflamatory effect of IL-1.

The following examples further illustrate practice of the present invention in demonstrating BMP promotion of chondrogenic differentiation of non-tissue culture expanded human mesenchymal precursor cells.

### EXAMPLE 1

Isolation and Culture Expansion of MMCs

Human MMCs were isolated according to previously reported procedure [Journal of Cellular Physiology 176, 57-669(1998)]. Mononuclear cells (MNCs) were isolated from human bone marrow samples according to a modification of a previously reported method [J Cell Physiol 185(1), 98 106(2000). Total nucleated cells in the marrow sample was diluted to a concentration of 7×10⁶ cells per ml with isolation buffer (calcium and magnesium free phosphate-buffered saline (PBS), 2% bovine serum albumin (BSA), 0.6% sodium citrate and 1% penicillin-streptomycin). Thirty to 35 ml of the diluted cell suspension was layered over 15 ml of Ficoll-Paque (Pharmacia, Piscataway, N.J.) and centrifuged at 800× g for 20 min. The MNCs were collected, counted, washed with magnetic-activated cell sorting (MACS) buffer (PBS with 0.5% BSA and 2 mM EDTA, pH 7.2) and resuspended in MACS buffer at 2-4×10⁸ cells per ml. 1×10⁸ MNCs were incubated with 0.2 ml of anti-human CD105 antibody-microbeads for 45 min at 4° C. and CD105⁺ cells were isolated using the MS⁺ columns (Miltenyi Biotec) according to the manufacturer's recommendation. The CD105⁻ cells were collected as the column eluate, while the CD105⁺ cells remained attached to the column. CD105⁺ cells were recovered from the column by removing it from the magnet and flushing out the cells with MACS buffer. CD105⁺ cells were plated in 185 cm² Nunclon Solo flasks (Nunc Inc., Naperville, Ill.) at a density of 5-7.5×10⁵ cells per flask and cultured in complete medium consisting of Alpha-MEM supplemented with 10% fetal bovine serum (FBS, Hyclone, Logan, Utah) and 1% antimycotic-antibiotic at 37° C. in 5% CO₂ in air. Medium was changed after 48 h and thereafter every 3-4 days. At day 14, cells were detached by incubation with 0.05% trypsin-EDTA and designated primary (p0) and replated for expansion at a density of 1×10⁶ cells per flask as passage 1 cells. The cells reached 90% of confluence in 6-7 days, after which they were either passaged as mentioned, used in other assays or stored in 90% FBS and 10% dimethyl sulphoxide in liquid nitrogen for future use. The cells used for this study were derived from passage 2 or passage 3. The CD105⁺ were designated MMCs as they are of mesenchymal origin and have multipotential differentiation capability.

### EXAMPLE 2

Culture of MMCs in Alginate

The MMCs were encapsulated in alginate according to a previously reported procedure [J Cell Physiol 185(1), 98-106(2000)]. Briefly, MMCs were detached and washed with wash buffer (0.15M NaCl, 25 mM Hepes, pH 7.0) and resuspended at a density of 25×10⁶ per ml in 1.2% alginate in wash buffer. Individual beads of the cell suspension were expressed through a 20-gauge needle into a solution containing 102 mM CaCl₂ and 25 mM Hepes (pH 7.0). The beads were allowed to polymerize for 10 min, washed once in wash buffer, three times in complete medium and cultured overnight in the same medium at 37° C. with 5% CO₂ in air. The next day, the medium was changed to chemically defined medium consisting of DMEM with high glucose, 100 nM dexamethasone (Sigma), 50 μg/ml ascorbic acid-2-phosphate (WAKO Pure Chemicals, Tokyo, Japan), 100 μg/ml of sodium pyruvate (Life Technologies), 50 μg/ml proline (Sigma), 1% ITS-Premix (Becton Dickenson, Bedford, Mass.) and 100 ng/ml rhBMP-2 or rhBMP-9. The medium was changed twice a week for the next 2-3 weeks. RNA was isolated from the cells in order to examine the expression of genes at various time intervals during the culture.

For IL-1 studies, MMCs were cultured in alginate for 14-21 days in the serum-free media with or without BMPs for chondrogenic differentiation. At day 14, beads were washed and cultured in the media with 200 pg/ml of IL-1 (Roche Biochemicals, Indianapolis, Ind.) for 72 h (day 17) as reported in a previous study [Journal of Cellular Physiology 176, 57-66(1998)]. The beads were again washed and cultured in media with BMPs for 96 h (day 21). RNA was isolated from the cells at days 14, 17 and 21. In the next study, MMCs were cultured in alginate beads for 14 days and subsequently for an additional 7 days in BMPs alone, IL-1 alone or in various combinations of IL-1 and BMPs together. RNA was isolated from the cells at days 14 and 21.

In another study the expanded cells were encapsulated in a 3-dimensional alginate matrix and cultured in serum free media with or without IL-11 and BMP-9 to analyze their potential to undergo chondrogenic differentiation.

### EXAMPLE 3

RNA Preparation and Northern Analysis

Culture-expanded MMCs were encapsulated in alginate beads and cultured in serum-free media. For RNA isolation, the beads were transferred to cell recovery buffer (55 mM Sodium Citrate, 0.15M NaCl and 25 mM Hepes, pH 7.0), incubated for 10 min at 4° C. to release the cells from the alginate matrix and centrifuged at 1400× g for 15 min at 4° C. to recover the cells. Total RNA was prepared from the cell pellet by a previously reported procedure [Journal of Cellular Physiology 176, 57-66(1998)]. Briefly, cell pellet was resuspended in lysis buffer (4M guanidinium isothiocynate, 0.03M sodium acetate and 0.4 g/ml of cesium chloride) and the lysate was layered over 5.7M cesium chloride and centrifuged for 18 h at 155,000× g in a SW40 rotor (Beckman, Palo Alto, Calif.). The RNA pellet was dissolved in water at 0.5-1 mg/ml. For northern blot analysis, 5 μg of total RNA per sample was fractionated on 1% formaldehyde-agarose gels. Subsequent to electrophoresis, RNA was transferred onto a positive charged nylon membrane, BrightStar-Plus (Ambion, Austin, Tex.). The gene probes for northern analysis was prepared as PCR amplified products using specific oligonucleotide primers as listed in Table I and the amplified products were confirmed by sequencing. These probes were radiolabeled by [α-³²P]dCTP (NEN Life Sciences Products) using the random primer method as recommended by the manufacturer (Amersham Pharmacia Biotech Inc., N.J.) and hybridized in ultrahyb solution (Ambion) overnight. Col2A1 hybridization was performed at 54° C. and all others were performed at 42° C. The filters were washed in 2× SSC/0.1% SDS at room temperature and then in 0.1× SSC/0.1% SDS at 65° C. for 30 min. The filter was exposed to X-ray film overnight. Hybridization signals were quantified by scanning the x-ray image and utilizing Image Gauge (Fuji Photo and Film Co, Japan). Col2A1, aggrecan, COMP and Sox-9 MRNA levels were corrected for RNA loading by normalization with β2-microglobulin. For detection of Col2A1 gene expression by reverse transcriptase-polymerase chain reaction (RT-PCR), RNA was prepared from cells isolated from 2-3 solubilized beads by the RNeasy kit (Qiagen, Valencia, Calif.). RT-PCR was performed using total RNA as a template, oligonucleotide primers, RNA PCR core kit (Perkin-Elmer, Norfolk, Conn.). The amplified products were analyzed on a 1.2% E-gels (Invitrogen, Carlsbad, Calif.).

TABLE I
Oligonucleotide primers used for PCR
amplification of probes

| Oligonucleotide primers/ | Size | |
|---|---|---|
| Reference/ | (bp) | |
| Human BETA2-MICROGLOBULIN | 270 | |
| | Majumdar et al | |
| Sense: 5′-TCTGGCCTTGAGGCTATCCAGCGT-3′ | (1998) | |
| Antisense: 5′-GTGGTTCACACGGCAGGCATACTC-3′ | | |
| Human COL2A1 | 451 | X16468 |
| Sense: 5′-AACCTGGACAGAGGGAAGC-3′ | | |
| Antisense: 5′-GGGGCCAGGATTCCATTAC-3′ | | |
| Human AGGRECAN | 450 | M55172 |
| Sense: 5′-TACTCTGGGTTTTCGTGACTC-3′ | | |
| Antisense: 5′-CGATGCCTTTCACCACGACTT-3′ | | |
| Human Sox-9 | 381 | Z46629 |
| Sense: 5′-CCCGATCTGAAGAAGGAGAGC-3′ | | |
| Antisense: 5′GTTCTTCACCGACTTCCTCCG-3′ | | |
| Human COMP | 501 | L32137 |
| Sense: 5′-GCAGATGCTTCGGGAACTGCA-3′ | | |
| Antisense: 5′-TTGATGCACACGGAGTTGGGG-3′ | | |

### EXAMPLE 4

BMP-2 and BMP-9 Induce Chondrogenic Differentiation of MMCs

Mesenchymal stem and progenitor cells cultured in the presence of TGF-β undergo chondrogenic differentiation [Tissue Engineering 4, 415-428(1998); The Journal of Bone and Joint Surjery 80(12), 1745-1757(1998)]. MMCs cultured in alginate and stimulated by TGF-β3 express Col2A1 and differentiate along the chondrogenic lineage [J Cell Physiol 185(1), 98 106(2000)]. To examine the effect of BMPs on chondrogenic differentiation, monolayer culture-expanded MMCs from 3 donors were further cultured in a 3-dimensional alginate matrix in the presence of 100 ng/ml of rhBMP-2 or rhBMP-9. RT-PCR was performed on RNA extracted from cells at various time intervals to detect the expression of Col2A1. The results showed that Col2A1 expression was induced in cells in all the 3 donors between day 8 and day 14. At day 14, total RNA was prepared from the cells in culture and northern blot analysis was performed. The results showed that all 3 donors responded to stimulation by both BMPs and expressed Col2A1, while the expression was undetected in untreated cells. The results also showed that BMP-9 treatment induced a higher level of Col2A1 expression than BMP-2.

It has been shown in mouse studies that BMP-2 upregulated the expression of chondrogenic-related transcription factor Sox-9 which in turn regulates the expression of Col2A1 and aggrecan [The Journal of Bone and Joint Surgery 82-A(2), 151-160(2000)]. To evaluate this observation in human MMCs and to further analyze the state of chondrogenic lineage progression, the expression of chondrogenic specific markers including aggrecan, cartilage oligomeric matrix protein (COMP) and Sox-9 was investigated. MMCs from multiple donors were analyzed and the results were from one donor, representative of the group. The expression of both aggrecan and COMP increased in response to BMPs in a manner similar to Col2A1 expression. Sox-9 showed a basal level of expression in the untreated cells, but underwent an observable upregulation in cells treated with BMPs. Therefore, it is contemplated that MMCs in alginate cultures are induced to differentiate along the chondrogenic lineage by BMP-2 and BMP-9.

In the I1-11 and BMP-9 study cells cultured without IL-11 or BMP-9 and with IL-11 alone did not express Col2A1. Cells cultured with increasing concentration of BMP-9 showed a significant level of Col2A1 expression when compared to untreated cells. Cells cultured in combination of IL-11 and BMP-9 showed higher levels of Col2A1 expression than BMP-9 alone. The synergistic effect of IL-11 and BBMP-9 was optimum at concentration of 10 ng/ml of I1-11 where the increase in Col2A1 expression was over 5-fold greater than BMP-9 alone.

### EXAMPLE 5

BMP Induction of a Steady Increase in Expression of the Chondrogenic Markers in MMCs in a Time-Dependant Manner

RNA isolated from MMCs cultured in alginate beads at day 5, 10 and 15 were subjected to northern analysis. The results showed (FIG. 1) that Col2A1 gene expression was detected at day 10 with a sequential increase at day 15. Aggrecan expression was detected at day 5 and showed a progressive increase with days in culture. Again BMP-9 treated cells showed higher expression of both Col2A1 and aggrecan than BMP-2 treated cells. The results indicate that aggrecan expression responds earlier than Col2A1 expression when MMCs are treated with BMP-2 and BMP-9.

### EXAMPLE 6

BMPs Overcome the Inflammatory Effect of IL-1

Previous studies have shown that IL-1 inhibits chondrogenic specific genes including Col2A1 and aggrecan by downregulation of the transcription factor Sox-9 Biochim Biophys Acta 1052(3), 366-78(1990); J Cell Physiol 166(2), 351-9(1996); J Biol Chem 275(5), 3687-92(2000)]. The effect of IL-1 on chondrogenic differentiated MMCs was examined by analyzing the expression of these three genes. The results showed that at day 14, BMP-2 and BMP-9 induced expression of Col2A1 and aggrecan and, as mentioned before, BMP-9 treatment caused a higher level of expression than BMP-2 treatment (FIG. 2, lanes 1-3). Removal of BMPs and addition of IL-1 for 72 h led to a reduced level of expression of Col2A1, aggrecan and Sox-9 (lanes 4-6). Removal of IL-1 and addition of BMPs for an additional period of 96 h resulted in rebound expression of the three genes (lanes 7-9) with the expression level similar to cells continuously exposed to BMPs for 21 days (lanes 10-12).

The effect of BMPs in the presence of IL-1 was then examined. Cells were allowed to differentiate along the chondrogenic lineage for 14 days followed by 7 days in the presence of IL-1 alone, BMPs alone, and IL-1 and BMPs together. The results showed (FIG. 3) that untreated cells (lane 1) and 14 days untreated cells exposed to an increasing concentration of IL-1 for an additional 7 days showed no Col2A1 expression and no appreciable expression of Sox-9 (lanes 2-4) as expected. Cells treated with BMPs progressed through chondrogenic differentiation during the first 14 days as demonstrated by Col2A1 expression (lanes 5 and 18). Exposure of the chondrocytic-differentiated cells to an increasing concentration of BMP-2 for an additional 7 days (lanes 6-8) increased expression of Col2A1 and Sox-9. A similar effect was observed with BMP-9 treatment (lanes 19-21), although maximal response to BMP-9 was achieved at the lowest dose of 100 ng/ml. Both BMP-2 and BMP-9 were able to partially prevent the IL-1 induced suppression of Col2A1 and Sox-9 (lanes 9-17 and 22-30 respectively) especially at the lowest concentration of IL-1 used (20 pg/ml). In addition, BMP-9 was able to maintain a higher level of Col2A1 expression at all concentrations of IL-1. These observations showed that BMPs are potent molecules that have the ability to function as anabolic factors in an environment containing inflammatory cytokines.

### EXAMPLE 7

Isolation and Culture Expansion of CD105⁺ cells

Human MMCs were isolated according to a previously reported procedure [Majumdar et al., Journal of Cellular Physiology. 185:98-106 (2000)]. Mononuclear cells (MNCs) were isolated from human bone marrow, washed with magnetic-activated cell sorting (MACS) buffer consisting of phosphate buffered saline with 0.5% BSA and 2 mM EDTA, pH 7.2. The cells were resuspended in MACS buffer and 1×10⁸ MNCs were incubated with 0.2 ml of anti-human CD105 antibody-microbeads (Miltenyi Biotec, Auburn, Calif.) for 45 min at 4° C. The cells were then washed and separated on a magnetic column MS⁺ (Miltenyi Biotec) according to the manufacturer's recommendation. The column eluate consisted of the CD105⁻ cells. The attached CD105⁺ cells were recovered by removing the column from the magnet and flushing out the cells with MACS buffer. A small fraction (5-7.5×10⁵) of the CD105⁺ cells were plated in 185 cm² Nunclon Solo flasks (Nunc Inc., Naperville, Ill.) in complete medium consisting of Alpha-MEM supplemented with 10% fetal bovine serum (FBS, Hyclone, Logan, Utah) and 1% antimycotic-antibiotic (Life Technologies, Gaithersburg, Md.) at 37° C. in 5% CO₂ in air to analyze for the plating efficiency of the cells. At day 14, cells were detached by 0.05% trypsin-EDTA (Life Technologies) treatment and stored in 90% FBS and 10% dimethyl sulphoxide in liquid nitrogen for future use.

### EXAMPLE 8

### Flow Cytometry

Analysis of cell surface molecules was performed according to a previously reported procedure [Majumdar et al., Journal of Cellular Physiology. 176:57-66. (1998)]. Column-isolated CD105⁺ cells were washed in FACS buffer (2% BSA, 0.1% sodium azide in PBS) and aliquots (1×10⁵-1×10⁶) of cells were incubated with anti-human CD45 fluorochrome-conjugated monoclonal antibodies (Pharmingen, San Diego). Cells were washed and the cell pellet was resuspended in FACS buffer with 1% paraformaldehyde. Nonspecific fluorescence was determined using equal aliquots of cell preparation that were incubated with mouse isotype monoclonal antibodies. Data were collected by analyzing 10,000-50,000 events on a Becton Dickson instrument (San Jose, Calif.) using Cell-Quest software.

### EXAMPLE 9

### Culture of CD105⁺ Cells in Alginate

The CD105⁺ cells were encapsulated in alginate by modification of a previously reported procedure (Majumdar et al., 2000). Cells were washed with wash buffer (0.15M NaCl, 25 mM Hepes, pH 7.0) and resuspended at a density of 10-20×10⁶ per ml in 1.2% alginate in wash buffer. Individual beads of the cell suspension were then slowly expressed through a 20-gauge needle into a solution containing 102 mM CaCl₂ and 25 mM Hepes (pH 7.0). The beads were allowed to polymerize for 10 min, washed once in wash buffer, three times in complete medium and cultured overnight in the same medium at 37° C. with 5% CO₂ in air. The next day, the medium was changed to chemically defined medium (Majumdar et al., 2000). The alginate beads were cultured in the above medium (untreated) or medium supplemented with 100 ng/ml BMP-2, or BMP-9 (treated). The medium was changed twice a week for the next 3 weeks. MNCs as well as CD105⁻ cells were also encapsulated in alginate beads at the same cell concentration and cultured similarly.

### EXAMPLE 10

### RNA Preparation and Analysis

CD105⁺ cells from multiple donors were encapsulated in alginate beads and cultured for 3 weeks. At the end of the culture period, cells were recovered from the beads, total RNA was extracted from the cell pellet by RNeasy kit (Qiagen, Valencia, Calif.) and reverse transcriptase-polymerase chain reaction (RT-PCR)-elisa was performed according to a previously reported procedure (Majumdar et al., 1998). Briefly, RT-PCR was performed using total RNA as a template, oligonucleotide primers (Table II), RNA PCR core kit (Perkin-Elmer, Norfolk, Conn.), and substituting the deoxy-nucleotides with digoxigenin-labeled nucleotides (Roche Biochemicals, Indianapolis, Ind.) to label the amplified products. Elisa was performed as recommended by the manufacturer (Roche Biochemicals, Indianapolis, Ind.). The data for each untreated and treated sample from each donor were normalized to b2-microglobulin. Progression was determined by cartilage specific markers including type II collagen, aggrecan and link protein. For each donor the expression of type II collagen, aggrecan and link protein was computed as fold increase over untreated. The result shown is the mean fold increase for 3 donors. RT-PCR elisa analysis (FIG. 4) showed that in comparison to untreated cells, BMP-2 and BMP-9 treated cells had a significant increase in gene expression for chondrogenic specific genes including Col2A1, aggrecan and link protein suggesting that the CD105+ cells were undergoing chondrogeic differentiatiton. In contrast, MNCs as well as CD105− cells did not show any evidence of chondrogenic differentiation.

TABLE II
Oligonucleotide primers for RT-PCR elisa.

| Oligonucleotide primers/ | Size | Reference/ |
|---|---|---|
| 5′-biotinylated probes | (bp) | Accession # |
| Human BETA2-MICROGLOBULIN | 270 | Majumdar et al. |
| Sense: 5′-TCTGGCCTTGAGGCTATCCAGCGT-3′ | | (1998) |
| Antisense: 5′-GTGGTTCACACGGCAGGCATACTC-3′ | | |
| Probe: 5′-Biotinylated CATCCATCCGACATTGAAGTTGAC-3′ | | |
| Human COL2A1 | 451 | X16468 |
| Sense: 5′-TCCCAAAGGTGCTCGAGGAGA-3′ | | |
| Antisense: 5′-CTCACCACGATCACCCTTGAC-3′ | | |
| Probe: 5′-Biotinylated GAGAGAGGATTCCCTGGCTT-3′ | | |
| Human AGGRECAN | 450 | M55172 |
| Sense: 5′-TACTCTGGGTTTTCGTGACTC-3′ | | |
| Antisense: 5′-CGATGCCTTTCACCACGACTT-3′ | | |
| Probe: 5′-Biotinylated GAGAAGGAGGTAGTGCTGCT | | |
| Human LINK PROTEIN | 361 | X17405 |
| Sense: 5′-GCTGATTTCAATCTGCTGGG -3′ | | |
| Antisense: 5′-GTCTGTGATGACCAGAGAAGC-3′ | | |
| Probe: 5′-Biotinylated AGCATTTGGCTCAGGAATCC-3′ | | |

### EXAMPLE 11

### Immunohistochemistry

Immunohistochemistry was performed to detect the presence of type II collagen protein in the alginate according to a previously reported procedure (Majumdar et al., 2000). Alginate beads from cultures were washed with water and incubated in 100 mM barium chloride for 10 min for irreversible polymerization. The beads were then washed with water again and fixed in 10% buffered formalin and embedded in paraffin. Sections of alginate beads were incubated with goat anti-type II collagen antibody (Southern Biotechnology Associates, Birmingham, Ala.). Immunoreactivity was detected by incubating sections with biotinylated anti-goat antibody and horse radish peroxidase H reagents (Vector Laboratories, Burlingame, Calif.). Signal was developed by treating the sections with peroxidase substrate 3,3′-diaminobenzidine (DAB) and H₂O₂. Images were recorded on 35 mm slide film and multipanel figures were made with Photoshop (Adobe Systems, San Jose, Calif.). Experimental controls consisted of alginate sections stained with nonimmune primary antibody followed by secondary antibody. The results indicate that in comparison to the untreated cells, BMP-2 and BMP-9 treated cells showed a significant presence of type II collagen protein. Type II collagen protein was present in the intercellular region due to the secretion of the protein by the differentiating cells and subsequent entrapment in the alginate matrix. Alginate sections stained with nonimmune primary antibody did not show any immunoreactivity.

While this invention has been particularly shown and described with references to preferred embodiments thereof, it will be understood by those skilled in the art that various changes in form and details may be made therein without departing from the scope of the invention encompassed by the appended claims.

What is claimed is:

1. A composition for inducing cartilaginous tissue formation and maintenance comprising a BMP.

2. A method for inducing formation and/or maintenance of chondrocytes or cartilaginous tissue in a patient in need of same, said method comprising administering to said patient an effective amount of the composition of claim 1.

3. A method for treating arthritis, or other cartilaginous tissue defect in a patient in need of same, said method comprising administering to said patient an effective amount of the composition of claim 1.

4. A method for treating articular cartilage defects or damage in a patient in need of same, said method comprising administering to said patient an effective amount of the composition of claim 1.

5. The composition of claim 1, wherein said BMP is selected from the group consisting of BMP-2 and BMP-9.

6. The method of claim 2 wherein said BMP is selected from the group consisting of BMP-2 and BMP-9.

7. The method of claim 3 wherein said BMP is selected from the group consisting of BMP-2 and BMP-9.

8. The method of claim 4 wherein said BMP is selected from the group consisting of BMP-2 and BMP-9.

9. A composition for blocking or suppressing the inhibitory effects of IL-1 or TNF said composition comprising a BMP.

10. The composition of claim 9, wherein said BMP is selected from the group consisting of BMP-2 and BMP-9.

11. A method for blocking or suppressing the inhibitory effect of IL-1 comprising administering an effective amount of a BMP.

12. The method of claim 12 wherein said BMP is selected from the group consisting of BMP-2 and BMP-9.

13. A composition comprising MMCs and a BMP for use in articular cartilage repair.

14. A composition for inducing chondrogenesis said composition comprising IL-11 and BMP-9.

15. A composition for inducing chondrogenesis said composition comprising non-tissue culture expanded cells isolated from bone marrow.

16. The composition of claim 15 wherein said cells are CD105+ cells.

17. The composition of claim 16 further comprising a suitable matrix.

18. The composition of claim 17 further comprising a bone and/or cartilage inducing factor.

19. The composition of claim 18 wherein said factor is a BMP.

20. The composition of claim 19 wherein said BMP is selected from the group consisting of BMP-2 and BMP-9.

21. A method for inducing chondrogenesis in a patient in need of same, said method comprising administering to said patient an effective amount of a composition comprising non-tissue culture expanded cells isolated from bone marrow.

22. The method of claim 21 wherein said cells are CD105+ cells.

23. The method of claim 22 further comprising administering a bone and or cartilage inducing factor.

24. The method of claim 23 wherein said factor is a BMP.

25. The method of claim 24 wherein said BMP is selected from the group consisting of BMP-2 and BMP-9.

26. A method for treating arthritis, or other cartilaginous tissue defect in a patient in need of same, said method comprising administering to said patient an effective amount of the composition of claim 15.

27. A method for treating articular cartilage defects or damage in a patient in need of same, said method comprising administering to said patient an effective amount of the composition of claim 15.

28. A composition for tissue repair said composition comprising non-tissue culture expanded cells isolated from bone marrow and a bone and/or cartilage inducing factor.

29. The composition of claim 28 wherein said isolated cells are CD105+ cells and said factor is a BMP.

30. The composition of claim 29 further comprising a suitable matrix.

31. The composition of claim 30 wherein said BMP is selected from the group consisting of BMP-2 and BMP-9.
